(19) United States
(12) Patent Application Publication
(10) Pub. No.: US 2026/0108364 A1
(43) Pub. Date: Apr. 23, 2026

(54) ORTHOPAEDIC SURGICAL INSTRUMENT FOR SECURING AN EXTRACTION BLADE TO A POWER TOOL FOR EXTRACTION OF A FEMORAL STEM COMPONENT DURING A HIP REPLACEMENT SURGICAL PROCEDURE AND METHOD OF USING THE SAME

(71) Applicant: DePuy Ireland Unlimited Company, Ringaskiddy (IE)

(72) Inventors: Cory A. Shulaw, Warsaw, IN (US); Nathanael K. Conner, Fort Wayne, IN (US)

(21) Appl. No.: 19/325,424

(22) Filed: Sep. 10, 2025

Related U.S. Application Data
(60) Provisional application No. 63/709,682, filed on Oct. 21, 2024.

(51) Int. Cl. A61F 2/46

(52) U.S. Cl. CPC A61F 2/4607; A61F 2002/4619

(57) ABSTRACT

An orthopaedic surgical instrument for securing an extraction blade to a power tool during extraction of an implanted femoral stem component includes a clamping jaw configured to the clamp the extraction blade against the instrument's impact head. A leaf spring applies a spring bias to the clamped extraction blade. A method of using an orthopaedic surgical instrument to cut bone tissue away from an implanted femoral component is also disclosed.

This application claims priority under 35 U.S.C. § 119 to U.S. Provisional Patent Application Ser. No. 63/709,682 which was filed on Oct. 21, 2024 and is incorporated herein by reference in its entirety.

### TECHNICAL FIELD

The present disclosure relates generally to orthopaedic instruments for use in the performance of an orthopaedic joint replacement procedure, and more particularly to orthopaedic surgical instruments for use in the performance of a hip replacement procedure.

### BACKGROUND

Joint arthroplasty is a well-known surgical procedure by which a diseased and/or damaged natural joint is replaced by a prosthetic joint. The prosthetic joint may include a prosthesis that is implanted into one or more of the patient's bones. Many hip prostheses include a femoral prosthesis that is implanted into a patient's femur. A femoral prosthesis typically includes an elongated stem component that is installed in the intramedullary canal of the patient's femur and a spherically-shaped head component that bears against the patient's acetabulum or a prosthetic replacement acetabular cup.

Typical joint arthroplasty surgical procedures include impaction of surgical instruments (e.g., extraction blades, insertion/extraction instruments, broaches, or other cutting tools) and/or prosthetic implants into the patient's bone. In some surgical procedures, such as revision surgical procedures, it is necessary to remove a previously implanted femoral stem component. In such a case, one or more extraction blades may be used to cut the bone tissue away from the previously-implanted femoral stem component. Typically, the extraction blade is impacted by the surgeon to assert a cutting force on the extraction blade. Historically, such impaction has been performed by an orthopaedic surgeon manually striking a surgical instrument secured to the extraction blade using a surgical mallet or hammer.

Certain automated surgical impactors are capable of performing a series of percussive impacts that each provide a controlled amount of impaction force. An automated surgical impactor may be used with one or more adapters to connect to various surgical instruments and/or implants.

### SUMMARY

According to one aspect, an orthopaedic surgical instrument for securing an extraction blade to a power tool for extraction of a femoral stem component during an orthopaedic hip replacement surgical procedure on a patient's femur includes an elongated body having a connector formed in a proximal end of the elongated body. The connector is configured to fit into the chuck of an automated surgical impactor. The elongated body also includes an impact head formed in a distal end. The impact head has an impact surface that is configured to abut a connector of the extraction blade when the orthopaedic surgical instrument is used to impact the extraction blade. The instrument also includes a locking lever that has a pivot end pivotally coupled to the elongated body and an opposite latch end. A clamping jaw is slidably coupled to the elongated body. The clamping jaw is movable between a clamped position in which the clamping jaw is retracted toward the impact head, and a released position in which the clamping jaw is extended away from the impact head. The instrument also includes a leaf spring that has a first end pivotally coupled to the locking lever and a second end coupled to the clamping jaw.

In an embodiment, the impact surface of the impact head has an annular-shaped recess formed therein. The annular-shaped recess is sized and shaped to receive an annular-shaped collar of the connector of the extraction blade.

The clamping jaw may include a clamp head having a pair of arms extending proximally therefrom. The arms of the clamping jaw are coupled to the second end of the leaf spring.

The instrument may also include a pushbutton catch coupled to the elongated body. In such an embodiment, the locking lever is movable between an unlocked position in which the latch end is spaced apart from the elongated body and a locked position in which the latch end is captured by the pushbutton catch.

The instrument may also include a torsion spring positioned between the locking lever and the elongated body. The torsion spring biases the locking lever into its unlocked position.

In an embodiment, the leaf spring asserts a spring bias on the connector of the extraction blade when the connector of the extraction blade is positioned against the impact head and the locking lever is positioned in its locked position.

In an embodiment, the clamping jaw asserts a clamping force on the connector of the extraction blade when the connector of the extraction blade is positioned against the impact head and the locking lever is positioned in its locked position.

The second end of the leaf spring may be pivotally coupled to the clamping jaw.

According to another aspect, an orthopaedic surgical instrument assembly for extracting a femoral stem component during an orthopaedic hip replacement surgical procedure on a patient's femur includes an extraction blade and an adapter instrument. The extraction blade is configured to cut bone tissue to extract the femoral stem component from the patient's femur. The extraction blade has an elongated blade body that includes a cutting surface, and a connector extending proximally away from the elongated blade body. The adapter instrument is configured to secure the extraction blade to a power tool. The adapter instrument includes an elongated body having a connector formed in its proximal end. The connector is configured to fit into the chuck of an automated surgical impactor. An impact head is formed in a distal end of the elongated body. The adapter instrument also includes a locking lever having a pivot end pivotally coupled to the elongated body and an opposite latch end. A clamping jaw is slidably coupled to the elongated body. The clamping jaw is movable between a clamped position in which the clamping jaw is retracted toward the impact head so as to clamp the connector of the extraction blade between the clamping jaw and the impact head, and a released position in which the clamping jaw is extended away from the impact head.

In an embodiment, the adapter instrument further includes a leaf spring having a first end pivotally coupled to the locking lever and a second end coupled to the clamping jaw. The leaf spring asserts a spring bias on the connector of the extraction blade when the connector of the extraction blade is positioned against the impact head and the locking lever is positioned in its locked position.

In an embodiment, the connector of the extraction blade has an annular-shaped collar formed in its proximal end, and the impact surface of the adapter instrument's impact head has an annular-shaped recess formed therein. The annular-shaped recess is sized and shaped to receive the annular-shaped collar of the connector of the extraction blade.

In an embodiment, the clamping jaw of the adapter instrument includes a clamp head having a pair of arms extending proximally therefrom. The arms of the clamping jaw are coupled to the second end of the leaf spring.

In an embodiment, the adapter instrument further includes a pushbutton catch coupled to the elongated body. The locking lever is movable between an unlocked position in which the latch end is spaced apart from the elongated body and a locked position in which the latch end is captured by the pushbutton catch.

The adapter instrument may further include a spring positioned between the locking lever and the elongated body. The spring biases the locking lever into its unlocked position.

In an embodiment, the clamping jaw asserts a clamping force on the connector of the extraction blade when the connector of the extraction blade is positioned against the impact head and the locking lever is positioned in its locked position.

The second end of the leaf spring may be pivotally coupled to the clamping jaw.

According to another aspect, a method of extracting an implanted femoral stem component during performance of an orthopaedic hip replacement surgical procedure on a patient's femur includes positioning a connector of an extraction blade between an impact head and a clamping jaw of an adapter instrument, and thereafter moving a locking lever of the adapter instrument into a locked position so as to clamp the connector between the clamping jaw and the impact head of the adapter instrument. A connector of the adapter instrument is coupled to a chuck of an automated surgical impactor. The automated surgical impactor is operated to apply an impact force on the extraction blade.

In an embodiment, moving the locking lever of the adapter instrument into the locked position applies a spring bias on the connector of the extraction blade with a leaf spring.

In an embodiment, moving the locking lever of the adapter instrument into the locked position captures a latch end of the locking lever in a pushbutton catch so as to retain the locking lever in its locked position.

### BRIEF DESCRIPTION OF THE DRAWINGS

The detailed description particularly refers to the following figures, in which:

FIG. 1 is a side elevation view of an adapter instrument for use in an orthopaedic surgical hip replacement procedure on a patient's femur, note the instrument's locking lever is shown in its unlocked position in FIG. 1;

FIG. 2 is view similar to FIG. 1, but showing the instrument's locking lever positioned in its locked position;

FIG. 3 is a cross sectional view of the adapter instrument of FIG. 1 taken along the line 3-3 of FIG. 1, as viewed in the direction of the arrows;

FIG. 4 is a cross sectional view of the adapter instrument of FIG. 2 taken along the line 4-4 of FIG. 2, as viewed in the direction of the arrows;

FIG. 5 is a cross sectional view of the adapter instrument of FIG. 1 taken along the line 5-5 of FIG. 1, as viewed in the direction of the arrows;

FIG. 6 is a cross sectional view of the adapter instrument of FIG. 2 taken along the line 6-6 of FIG. 2, as viewed in the direction of the arrows;

FIG. 7 is a perspective view of the adapter instrument and the extraction blade;

FIG. 8 is an enlarged, fragmentary perspective view showing the connector of the extraction blade and the impact head of the adapter instrument in more detail;

FIG. 9 is a side elevation view showing the extraction blade positioned in the adapter instrument prior to locking of the locking lever;

FIG. 10 is a similar view to FIG. 9, but showing the extraction blade positioned in the adapter instrument after locking of the locking lever; and

FIG. 11 is a side elevation view showing the adapter instrument clamped to the extraction blade and coupled to an automated surgical impactor.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, proximal, distal, etcetera, may be used throughout the specification in reference to the orthopaedic implants and orthopaedic surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise. Additionally, it is to be understood that terms such as top, bottom, front, rear, side, height, length, width, upper, lower, and the like that may be used herein merely describe points of reference and do not necessarily limit embodiments of the present disclosure to any particular orientation or configuration.

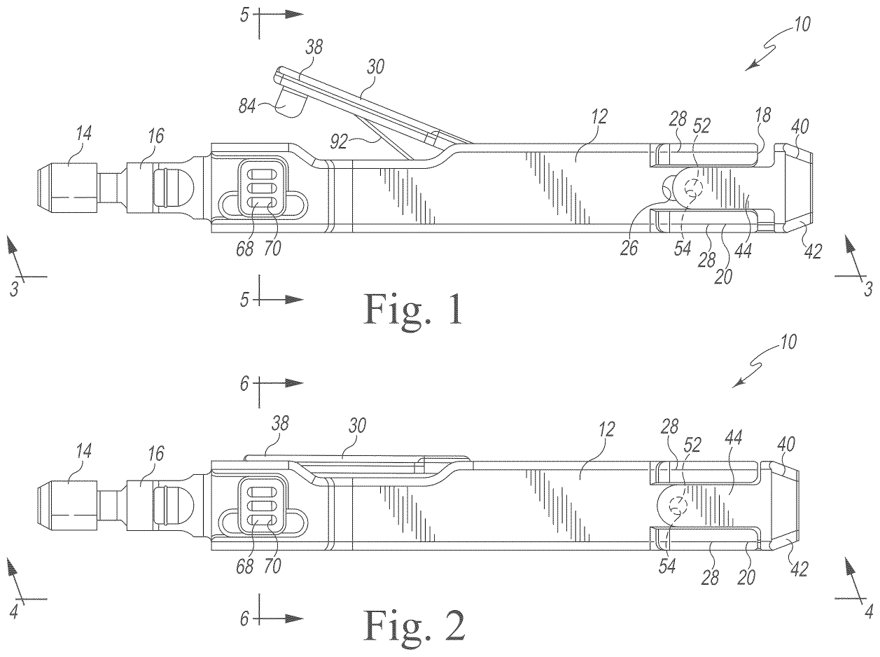

Referring now to FIGS. 1-6, an adapter instrument 10 for use with an automated surgical impactor for securing extraction blades to the automated surgical impactor is shown. The adapter instrument 10 is an orthopaedic surgical instrument; that is, a surgical tool used by a surgeon in performing an orthopaedic surgical procedure. As such, it should be appreciated that, as used herein, the terms “orthopaedic surgical instrument” and “orthopaedic surgical instruments” are distinct from orthopaedic implants or prostheses that are surgically implanted in the body of the patient. As described further below, the adapter instrument 10 is used with an automated surgical impactor to extract a previously-implanted femoral stem component from a patient's femur.

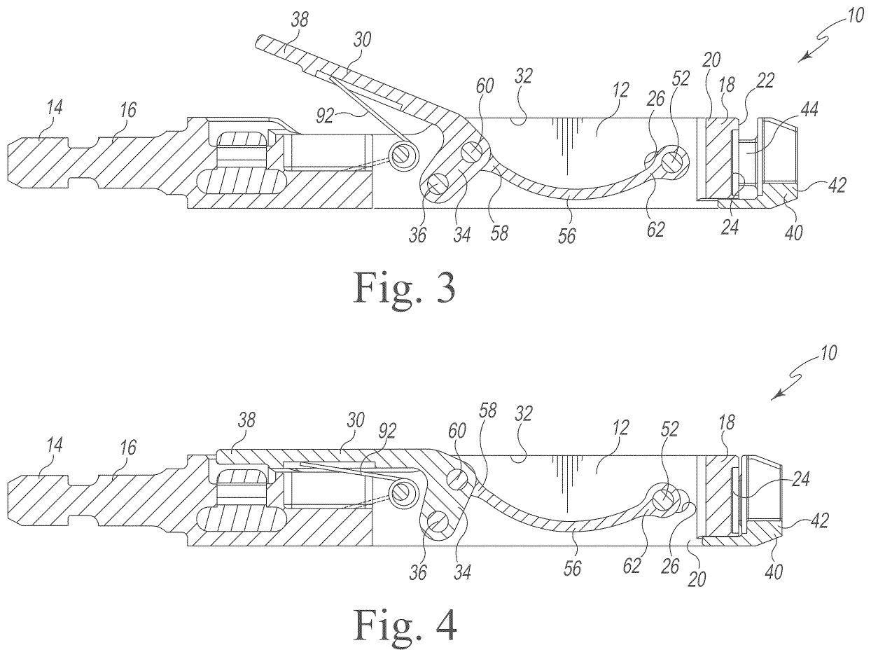

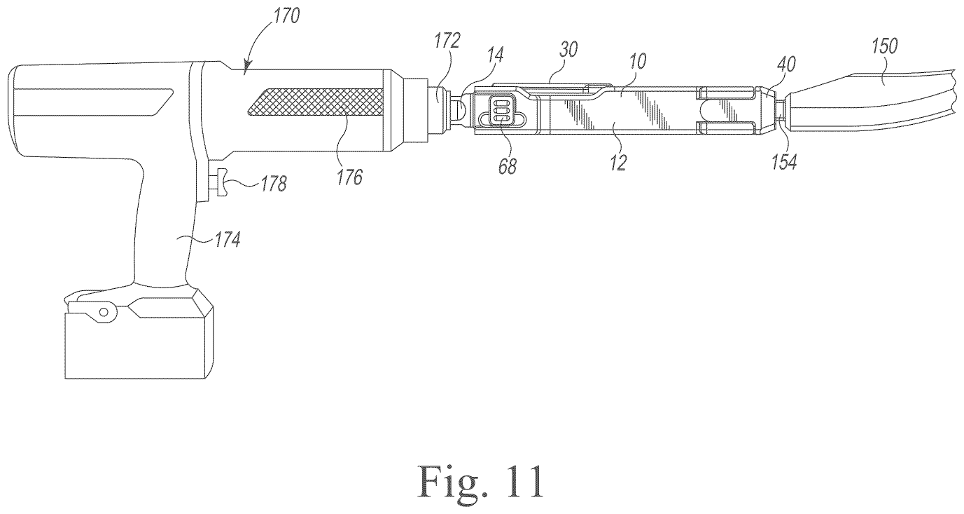

As shown in FIGS. 1-6, the adapter instrument 10 includes an elongated body 12 having a connector 14 formed in its proximal end 16. The connector 14 is configured to fit into the chuck of an automated surgical impactor (see FIG. 11). As shown best in FIGS. 3, 4, 7, and 8, an impact head 18 is formed in the distal end 20 of the instrument's elongated body 12. The impact head 18 has an impact surface 22 that has an annular-shaped recess 24 formed therein (see FIG. 8). The recess 24 of the impact head 18 is configured to receive, and thereafter capture therein, the collar 152 formed in the connector 154 of an extraction blade 150. It should be appreciated that although the recess 24 and the collar 152 are herein shown and described as being annular-shaped, other shapes are also contemplated for use. For example, the recess 24 and the collar 152 may be shaped as a square, rectangle, hexagon, or other polygon. Doing so would allow the rotational position of the extraction blade 150 relative to the adapter instrument 10 to be clocked in a number of repeatable rotational positions.

The adapter instrument 10 also includes an elongated locking lever 30 that extends outwardly from the inner cavity of the elongated body 12 through an opening 32 formed in the upper surface of the elongated body 12. The locking lever 30 includes a pivot end 34 that is pivotally coupled to the elongated body 12 via a pivot pin 36. As will be described below, an opposite, latch end 38 of the locking lever 30 is selectively captured by a pushbutton latch to retain the locking lever 30 in its locked position.

As shown in FIGS. 1-4, a clamping jaw 40 is slidably coupled to the elongated body 12. The clamping jaw 40 includes a clamp head 42 and a pair of arms 44 that extend proximally away from the clamp head 42. The clamp head 42 has an open slot 46 formed in its proximal side. As can be seen in FIG. 8, the slot 46 is shaped and sized to receive the collar 152 of the extraction blade's connector 154. The slot 46 is defined, in part, by a pair of proximally facing surfaces 48 which function as the clamp head's clamp surface 50. When the collar 152 of the extraction blade's connector 154 is positioned in the slot 46 and the locking lever 30 is positioned in its locked position, the collar 152 of the extraction blade's connector 154 is clamped between the clamp head's clamp surface 50 and the impact head's impact surface 22.

The clamping jaw 40 is configured to slide back and forth in a direction toward and a direction away from the impact head 18. Specifically, the clamping jaw 40 is movable between a clamped position and in which the clamping jaw is retracted toward the impact head 18 and a released position in which the clamping jaw 40 is extended away from the impact head 18. When the clamping jaw 40 is positioned in its clamped position, its clamp surface 50 is urged into contact with the collar 152 of the extraction blade 150 which, in turn, is urged into contact with the impact surface 22 of the impact head 18 so as to assert a clamping force on the collar 152. Such a clamping force captures the connector 154 of the extraction blade 150 by virtue of clamping the blade's collar 152 between the clamping jaw 40 and the impact head 18. Oppositely, when the clamping jaw 40 is positioned in its released position, the clamping jaw 40 is extended in a direction away from the impact head 18 thereby releasing the collar 152 of the extraction blade 150.

As shown in FIGS. 1-4, the proximal ends of the clamping jaw's arms 44 are coupled to one another by a pin 52. Specifically, each of the clamping jaw's arms 44 has a blind hole 54 formed in the inner surface of its proximal end. One end of the pin 52 is positioned in the hole 54 of one of the arms 44, with the opposite end of the pin 52 positioned in the hole 54 of the other arm 44. The elongated body 12 of the adapter instrument 10 has a pair of elongated slots 26 formed in its distal end near its impact head 18. One of the slots 26 is formed on each of the opposite sides of the elongated body 12. The pin 52 extends through the slots 26 (and the inner cavity of the elongated body 12) to couple the clamping jaw's arms 44 to one another. As will be described in more detail below, as the clamping jaw 40 is moved between extension and retraction (by use of the locking lever 30), the pin 52 translates back and forth within the slots 26.

As shown best in FIGS. 1, 2, 7, and 8, the elongated body 12 has a number of guide rails 28 formed on its outer surfaces. A pair of the guide rails 28 is formed on each side of the body's distal end near its impact head 18. Specifically, a pair of the guide rails 28 is formed on each of the opposite sides of the elongated body 12 such that one of the slots 26 is positioned between the individual rails 28 of the pair. Each of the clamping jaw's arms 44 is positioned between a pair of the guide rails 28. The width of the clamping jaw's arms 44 closely mimics the size of the gap between the guide rails 28 such the guide rails 28 guide and stabilize the clamping jaw's arms 44 during extension and retraction of the clamping jaw 40.

As can be seen in FIGS. 3 and 4, the pivot end 34 of the locking lever 30 is coupled to the clamping jaw 40 by a leaf spring 56. In particular, the proximal end 58 of the leaf spring 56 is pivotally coupled to the pivot end 34 of the locking lever 30 via a pivot pin 60, with the leaf spring's opposite distal end 62 being coupled to the pin 52 (and hence the arms 44 of the clamping jaw 40). The leaf spring 56 is utilized to put a fixating load in the form of a spring bias on the collar 152 of the extraction blade 150 thereby creating a rigid construct of the blade adapter 10 and the extraction blade 150. Such a fixating load is applied and removed by use of the locking lever 30. In particular, the locking lever 30 is movable between an unlocked position (as shown in FIGS. 1 and 3) in which the latch end 38 of the locking lever 30 is extended away from the elongated body 12 and a locked position (as shown in FIGS. 2 and 4) in which the latch end 38 is positioned proximate to the elongated body 12 and captured by a pushbutton catch 68. When the locking lever 30 is positioned in its unlocked position, the leaf spring 56 is relaxed and thus does not exert a spring bias on the clamping jaw 40 (and hence the collar 152 of the extraction blade 150). However, as the locking lever 30 is moved to its locked position, the leaf spring 56 is tensioned thereby exerting a spring bias on the clamping jaw 40 (and hence the collar 152 of the extraction blade 150).

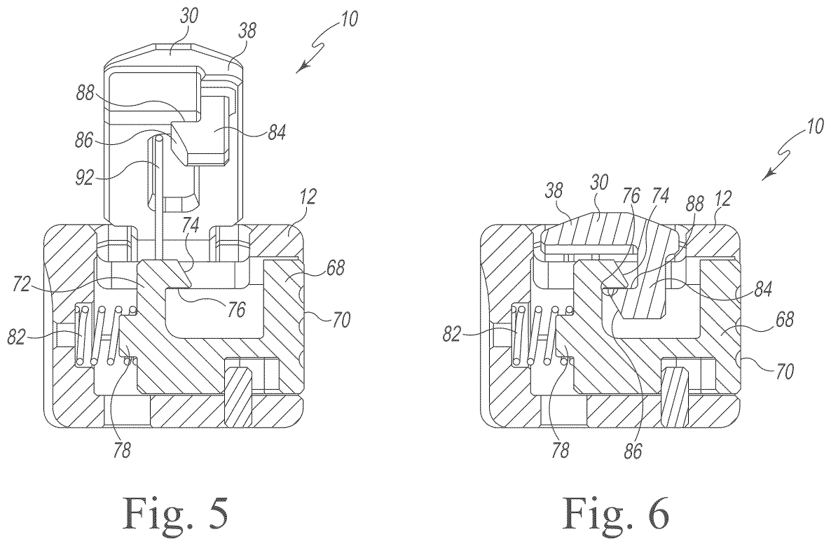

As alluded to above, the pushbutton catch 68 may be used to selectively retain the locking lever 30 in the locked position shown in FIGS. 2 and 4. As shown in FIGS. 5 and 6, the pushbutton catch 68 includes a button surface 70 positioned generally flush with the outer side surface of the elongated body 12. The button surface 70 is configured to be pressed by a surgeon and thus may be grooved or otherwise textured to provide enhanced grip.

As shown in FIGS. 5 and 6, the pushbutton catch 68 also includes a locking pawl 72 that includes a ramp-shaped upper cam surface 74 and a lower surface 76. A guide post 78 extends from a back surface of the locking pawl 72 toward an inner surface of the elongated body 12. A compression spring 82 is captured around the guide post 78 and thereby retained between the inner surface of the elongated body 12 and the back surface of the locking pawl 72. The spring 82 urges against the inner surface of the elongated body 12 and the back surface of the locking pawl 72 so as to bias the pushbutton catch 68 toward the outer surface of the elongated body 12 (i.e., the compression spring 82 urges the pushbutton catch 68 rightwardly as viewed in the orientation of FIGS. 5 and 6).

As shown in FIGS. 5 and 6, the latch end 38 of the locking lever 30 has a downwardly extending latch 84 formed therein. The latch 84 has a ramp-shaped lower cam surface 86 and an upper surface 88 formed therein. As shown in FIG. 6, when the locking lever 30 is in the locked position, the upper surface 88 of the latch 84 engages the lower surface 76 of the locking pawl 72 thereby retaining the latch 84. When the surgeon or other personnel depresses the button surface 70, the pushbutton catch 68 slides toward the opposite inner surface of elongated body 12 (i.e., it slides leftwardly as viewed in the orientation of FIGS. 5 and 6), and the lower surface 76 of the locking pawl 72 slides off the upper surface 88 of the latch 84 thereby releasing the locking lever 30. As can be seen in FIGS. 1, 3, 4, and 5, a torsion spring 92 biases the locking lever 30 into its unlocked position (note although the lower leg of the torsion spring 92 is not shown in the cross sections of FIGS. 3 and 5 as a result of the cut plane, it is shown in phantom for clarity of description). Thus, when the latch 84 is released by pressing the pushbutton catch 68, the latch end 38 of the locking lever 30 is urged toward its unlocked position by the spring bias of the torsion spring 92, which releases tension on the leaf spring 56.

When a surgeon or other user moves the lever 30 from its unlocked position to its locked position without depressing the pushbutton catch 68, the lower cam surface 86 of the latch 84 engages the upper cam surface 74 of the locking pawl 72. This engagement of the cam surfaces 74, 86 overcomes the spring bias of the compression spring 82 and forces the pushbutton catch 68 to slide toward the opposite inner surface of elongated body 12 (i.e., it slides leftwardly as viewed in the orientation of FIGS. 5 and 6). When the latch 84 passes the pawl 70 and the cam surfaces 74, 86 disengage, the spring 82 forces the pushbutton catch 68 to slide back toward its original position (i.e., the compression spring 82 urges the pushbutton catch 68 rightwardly as viewed in the orientation of FIGS. 5 and 6), which causes the upper surface 88 of the latch 84 to engage the lower surface 76 of the locking pawl 72 thereby retaining the latch 84.

As described above, use of the pushbutton catch 68 provides a positive lock on the locking lever 30 thereby preventing inadvertent release of the locking lever 30 during use of the adapter instrument 10. Moreover, the location of the pushbutton catch 68 in the elongated body 12 allows the locking lever 30 to be opened and/or closed by the surgeon using a single hand.

In the illustrative embodiment, the adapter instrument 10 is formed from a metallic material such as, for example, stainless steel. In particular, the elongated body 12, the locking lever 30, and the various internal components form an assembled metallic instrument. The adapter instrument 10 may be formed by conventional machining techniques, or alternatively, by the use of 3-D printing technology. In the case of 3-D printing, the adapter instrument 10 is formed in a layer-by-layer fashion.

As alluded to above, the extraction blade 150 is used to free a previously-implanted femoral stem component from a patient's femur during a revision hip replacement procedure. Specifically, as a result of bony ingrowth, an implanted femoral stem component becomes rigidly affixed to the patient's femur over time. During extraction of such a previously-implanted femoral stem component, it may be necessary to cut the bone tissue adjacent to the component - i.e., cut the ingrown bone tissue. Specifically designed extraction blades may be used to do so. Extraction blades are designed to fit around the profile of the implanted femoral stem component and cut along its outer surfaces. Specific blades for the medial and lateral sides of the femoral stem component may be used, along with specific blades based on the type of femoral stem component being extracted (e.g., straight versus bowed stem component). One example of a system of extraction blades is sold under the Watson Extraction System® which is commercially available from DePuy Synthes of Warsaw, Indiana.

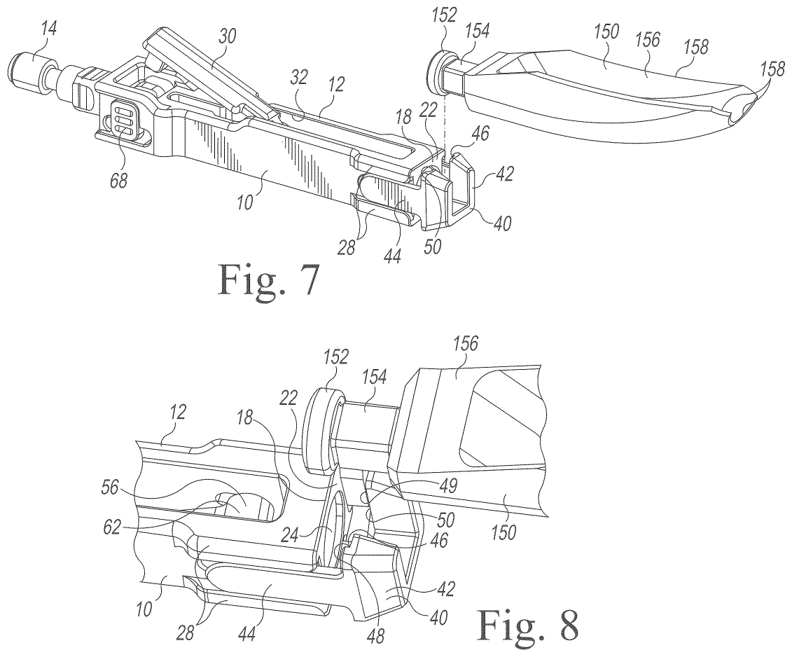

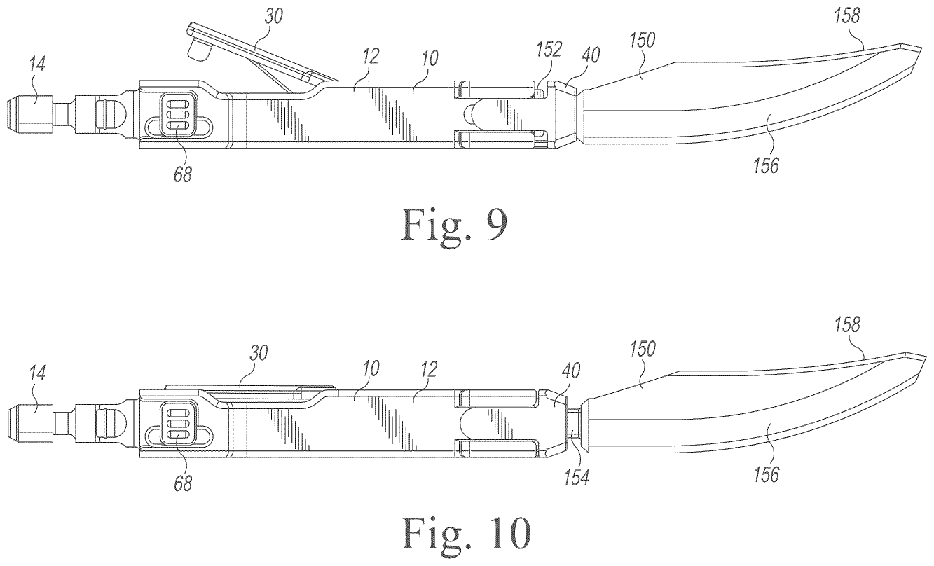

As shown in FIG. 7, the extraction blade 150 includes an elongated blade body 156. The blade body 156 of a given extraction blade 150 is shaped and sized to perform a particular cut along the implanted femoral stem component (e.g., medial side of the component, lateral side of the component, proximal body side of the component, etc). The blade body 156 includes a number of sharpened cutting surfaces 158 that cut bone tissue during advancement of the extraction blade 150 into the patient's femur. The blade's connector 154 extends proximally away from the blade body 156. As shown in FIGS. 7 and 8, the annular-shaped collar 152 is formed in the proximal end of the connector 154.

In use, the adapter instrument 10 may be used by a surgeon in conjunction with the extraction blade 150 to extract an implanted femoral stem component from the intramedullary canal of a patient's femur during a hip replacement surgical procedure such as a revision hip replacement surgical procedure. Prior to extraction of the previously-implanted femoral stem component, the surgeon performs a number of intra-operative surgical steps to gain access to the implanted femoral stem component. The surgeon also removes the femoral head component from the implanted femoral stem component's trunnion.

Thereafter, the surgeon assess the previously-implanted femoral stem component and selects one of the extraction blades 150 for use in cutting the stem component free of the ingrown bone tissue. The surgeon then assembles the selected extraction blade 150 to the adapter instrument 10. To do so, the surgeon positions the collar 152 of the selected extraction blade 150 in the slot 46 of the adapter instrument's clamp head 42 so as to position the collar 152 between the clamp head 42 and the instrument's impact head 18. Once the collar 152 of the extraction blade 150 is positioned between the clamp head 42 and the impact head 18, the surgeon moves the locking lever 30 into its locked position so as to move the clamping jaw 40 in the direction toward the impact head 18 thereby moving the clamping jaw 40 into its clamped position in which the collar 152 of the extraction blade 150 is securely captured (i.e., clamped) between the clamp head 42 and the impact head 18. Moving the locking lever 30 into its locked position applies a fixating load on the clamped extraction blade 150. Specifically, as the surgeon squeezes the adapter instrument 10 to urge the locking lever 30 into its locked position, its latch 84 is captured and retained by the pushbutton catch 68. Doing so tensions the leaf spring 56 which asserts a fixating load in the form of a spring bias on the collar 152 of the extraction blade 150 thereby creating a rigid construct of the instrument 10 and the extraction blade 150.

Thereafter, the adapter instrument's connector 14 is coupled to the chuck 172 of an automated surgical impactor 170 (see FIG. 11). The automated surgical impactor 170 may be embodied as a Kincise™ surgical automated system commercially available from DePuy Synthes of Warsaw, Indiana. In the illustrative embodiment, the automated surgical impactor 170 includes a chuck 172 in the form of a twist-lock collar. The automated surgical impactor 170 also includes a primary hand grip 174, a secondary hand grip 176, and a trigger 178. Once the chuck 172 has been coupled to the connector 14 of the adapter instrument 10, the surgeon positions the extraction blade 150 around the previously-implanted femoral stem component and depresses the trigger 178, which causes the automated surgical impactor 170 to generate an impact force in the form of a series of controlled percussive impacts on the adapter instrument 10. The adapter instrument 10 communicates the impact force from those percussive impacts to the extraction blade 150 thereby driving the blade's cutting surfaces 158 into the bone tissue of the patient's femur surrounding the femoral stem component. During such bone cutting, the surgeon's hands may remain on the automated surgical impactor 170 since the pushbutton catch 68 maintains the locking lever 30 in its locked position. Additionally, the leaf spring 56 retains the extraction blade 150 rigidly secured to the adapter instrument 10 during impaction. Unlike adapters using a typical rigid drive train attachment mechanism, the compliant, flexible leaf spring 56 of the adapter instrument 10 may not back out or otherwise loosen during impaction, even when subject to frequent, lower-amplitude impactions generated by the automated surgical impactor 170. It should be appreciated that a number of different extraction blades 150 may be used in progressive fashion to remove the bone tissue from the outer surfaces of the femoral stem component.

Once the surgeon has cut the surrounding bone tissue away from the previously-implanted femoral stem component, the connector 14 of the stem insertion instrument 10 may be decoupled from the chuck 172 of the automated surgical impactor 170. The surgeon or other personnel may then depress the button surface 70 of the pushbutton catch 68 so as to release the locking lever 30 thereby causing the locking lever 30 to be automatically moved to its unlocked position (by the spring bias asserted on it). Doing so moves the clamping jaw 40 in the direction away from the impact head 18 thereby moving the clamping jaw 40 into its released position in which the collar 152 of the extraction blade 150 is released from the adapter instrument 10.

With the surrounding bone tissue having been cut away from the outer surfaces of the previously-implanted femoral stem component, the stem component may be extracted from the intramedullary canal of the patient's femur. Thereafter, the surgeon performs the remaining steps in the surgical procedure.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic surgical instrument for securing an extraction blade to a power tool for extraction of a femoral stem component during an orthopaedic hip replacement surgical procedure on a patient's femur, the orthopaedic surgical instrument comprising:
an elongated body having (i) a connector formed in a proximal end of the elongated body, the connector being configured to fit into the chuck of an automated surgical impactor, and (ii) an impact head formed in a distal end of the elongated body, the impact head having an impact surface that is configured to abut a connector of the extraction blade when the orthopaedic surgical instrument is used to impact the extraction blade,
a locking lever having a pivot end pivotally coupled to the elongated body and an opposite latch end,
a clamping jaw slidably coupled to the elongated body, the clamping jaw being movable between (i) a clamped position in which the clamping jaw is retracted toward the impact head, and (ii) a released position in which the clamping jaw is extended away from the impact head, and
a leaf spring having a first end pivotally coupled to the locking lever and a second end coupled to the clamping jaw.

2. The orthopaedic surgical instrument of claim 1, wherein:
the impact surface of the impact head has an annular-shaped recess formed therein, and
the annular-shaped recess is sized and shaped to receive an annular-shaped collar of the connector of the extraction blade.

3. The orthopaedic surgical instrument of claim 1, wherein:
the clamping jaw includes a clamp head having a pair of arms extending proximally therefrom, and
the arms of the clamping jaw are coupled to the second end of the leaf spring.

4. The orthopaedic surgical instrument of claim 1, further comprising a pushbutton catch coupled to the elongated body, wherein the locking lever is movable between an unlocked position in which the latch end is spaced apart from the elongated body and a locked position in which the latch end is captured by the pushbutton catch.

5. The orthopaedic surgical instrument of claim 4, further comprising a torsion spring positioned between the locking lever and the elongated body, the torsion spring biases the locking lever into its unlocked position.

6. The orthopaedic surgical instrument of claim 1, wherein the leaf spring asserts a spring bias on the connector of the extraction blade when the connector of the extraction blade is positioned against the impact head and the locking lever is positioned in its locked position.

7. The orthopaedic surgical instrument of claim 1, wherein the clamping jaw asserts a clamping force on the connector of the extraction blade when the connector of the extraction blade is positioned against the impact head and the locking lever is positioned in its locked position.

8. The orthopaedic surgical instrument of claim 1, wherein the second end of the leaf spring is pivotally coupled to the clamping jaw.

9. An orthopaedic surgical instrument assembly for extracting a femoral stem component during an orthopaedic hip replacement surgical procedure on a patient's femur, the orthopaedic surgical instrument assembly comprising:
an extraction blade configured to cut bone tissue to extract the femoral stem component from the patient's femur, the extraction blade comprising (i) an elongated blade body that includes a cutting surface, and (ii) a connector extending proximally away from the elongated blade body, and
an adapter instrument configured to secure the extraction blade to a power tool, the adapter instrument comprising:
an elongated body having (i) a connector formed in a proximal end of the elongated body, the connector being configured to fit into the chuck of an automated surgical impactor, and (ii) an impact head formed in a distal end of the elongated body,
a locking lever having a pivot end pivotally coupled to the elongated body and an opposite latch end, and
a clamping jaw slidably coupled to the elongated body, the clamping jaw being movable between (i) a clamped position in which the clamping jaw is retracted toward the impact head so as to clamp the connector of the extraction blade between the clamping jaw and the impact head, and (ii) a released position in which the clamping jaw is extended away from the impact head.

10. The orthopaedic surgical instrument assembly of claim 9, wherein the adapter instrument further comprises a leaf spring having a first end pivotally coupled to the locking lever and a second end coupled to the clamping jaw.

11. The orthopaedic surgical instrument assembly of claim 9, wherein:
the connector of the extraction blade has an annular-shaped collar formed in its proximal end,
the impact surface of the adapter instrument's impact head has an annular-shaped recess formed therein, and
the annular-shaped recess is sized and shaped to receive the annular-shaped collar of the connector of the extraction blade.

12. The orthopaedic surgical instrument assembly of claim 9, wherein:
the clamping jaw of the adapter instrument includes a clamp head having a pair of arms extending proximally therefrom, and
the adapter instrument further comprises a leaf spring having a first end pivotally coupled to the locking lever and a second end coupled to the arms of the clamping jaw.

13. The orthopaedic surgical instrument assembly of claim 9, wherein:
the adapter instrument further comprises a pushbutton catch coupled to the elongated body, and
the locking lever is movable between an unlocked position in which the latch end is spaced apart from the elongated body and a locked position in which the latch end is captured by the pushbutton catch.

14. The orthopaedic surgical instrument assembly of claim 13, wherein:
the adapter instrument further comprises a spring positioned between the locking lever and the elongated body, and
the spring biases the locking lever into its unlocked position.

15. The orthopaedic surgical instrument assembly of claim 9, wherein:
the adapter instrument further comprises a leaf spring having a first end pivotally coupled to the locking lever and a second end coupled to the clamping jaw, and
the leaf spring asserts a spring bias on the connector of the extraction blade when the connector of the extraction blade is positioned against the impact head and the locking lever is positioned in its locked position.

16. The orthopaedic surgical instrument assembly of claim 9, wherein the clamping jaw asserts a clamping force on the connector of the extraction blade when the connector of the extraction blade is positioned against the impact head and the locking lever is positioned in its locked position.

17. The orthopaedic surgical instrument assembly of claim 9, wherein the adapter instrument further comprises a leaf spring having a first end pivotally coupled to the locking lever and a second end pivotally coupled to the clamping jaw.

18. A method of extracting an implanted femoral stem component during performance of an orthopaedic hip replacement surgical procedure on a patient's femur, comprising:
positioning a connector of an extraction blade between an impact head and a clamping jaw of an adapter instrument,
moving a locking lever of the adapter instrument into a locked position so as to clamp the connector between the clamping jaw and the impact head of the adapter instrument,
coupling a connector of the adapter instrument to a chuck of an automated surgical impactor, and
operating the automated surgical impactor to apply an impact force on the extraction blade.

19. The method of claim 18, wherein moving the locking lever of the adapter instrument comprises moving the locking lever of the adapter instrument into the locked position so as to apply a spring bias on the connector of the extraction blade with a leaf spring.

20. The method of claim 18, wherein moving the locking lever of the adapter instrument into the locked position comprises capturing a latch end of the locking lever in a pushbutton catch so as to retain the locking lever in its locked position.
